(19) United States
(12) Patent Application Publication
(10) Pub. No.: US 2015/0150704 A1
(43) Pub. Date: Jun. 4, 2015

(54) WEARABLE ROBOTIC DEVICE WITH BRACING SYSTEM WITH MOISTURE AND PRESSURE MANAGEMENT FOR COMFORTABLE REHABILITATION

(71) Applicant: The Hong Kong Polytechnic University, Hong Kong (HK)

(72) Inventors: Xiaoling Hu, Hong Kong (HK); Kai Yu Tong, Hong Kong (HK); Peter Man Kit Pang, Hong Kong (HK); Junyan Hu, Hong Kong (HK); Qing Ye, Hong Kong (HK); Shu To Ng, Hong Kong (HK); Junbo Tu, Hong Kong (HK); Shaojie Zhang, Hong Kong (HK); Waiming Li, Hong Kong (HK); Wai Honwah, Hong Kong (HK); Wei Rong, Hong Kong (HK)

(73) Assignee: The Hong Kong Polytechnic University, Hong Kong (HK)

(21) Appl. No.: 14/093,066

(22) Filed: Nov. 29, 2013

(51) Int. Cl. A61F 5/01

(52) U.S. Cl. CPC A61F 5/0102; A61F 2005/0174; A61F 2005/0134

(57) ABSTRACT

A wearable robotic device for the rehabilitation training of a limb with moisture and pressure management functions. Such wearable robotic device comprises a motor rotation system and a bracing system, wherein the motor rotation system comprises a motor and a motor control system; and the bracing system comprises a framework of structural members that is attachable to and detachable from the limb, one or more textile bracing cushions and one or more fastening belts attached to each of the textile bracing cushions such that the fastening belts and the textile bracing cushions in combination are capable of attaching the wearable robotic device onto the limb.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The presently claimed invention relates generally to a wearable robotic device for rehabilitation purposes, and more specifically relates to the mechanism for attaching the wearable robotic device onto a limb of the user.

### BACKGROUND

For patients who are suffering from motor impairments due to injuries or other medical conditions, such as stroke, rehabilitation treatment plays an important role in helping them recover muscular control on their affected limbs. Rehabilitation treatment generally involves repeated exercising of the patient's affected limb, and usually assistance is needed for the patient to carry out the required movements. During a rehabilitation session, such assistance is normally provided by a therapist. However, there might be occasions where the assistance required is beyond the strength that can be provided by a person of normal fitness. Further, the patient may sometimes wish to carry out the rehabilitation exercises outside of the rehabilitation sessions, and this is where rehabilitation robotics comes in.

Rehabilitation robotics refers broadly to the field of research that is dedicated to the understanding and enhancement of rehabilitation through the application of robotic devices, and its scope includes the development of wearable robotic devices for rehabilitation purposes. Wearable robotic devices are portable systems that can be used by the patient alone to carry out certain rehabilitation exercises (according to the design of the device) and can be directly worn onto a physically-impaired limb. They usually include a structural framework for attaching the device onto the user's limb and a motor having an external power supply to execute movements of the joint. Some examples of this type of device are disclosed in U.S. Pat. Nos. 7,056,297 and 8,556,836 and U.S. Pat. App. Pub. Nos. 2011/0112447 and 2011/0251533.

One of the major problems with this kind of device is that slippage can easily occur around the contacting areas between the limb and the device during physical training to such extent that the motor becomes misaligned with the joint and hence the rotations of the motor cannot be effectively converted to movements of the joint.

One of the reasons for the slippage is that during physical training, the changes in the circumference of the limb, due to muscle contraction or relaxation, together with the weight of the device can easily cause the device to slide along the limb. Another reason is that the perspiration released during the movements of the limb lubricates the contacting areas between the skin surface of the limb and the device, and that further exacerbates the slippage of the device.

An obvious solution would be to tightly strap the device onto the user's limb with a fastening belt. However, this poses the risk of having the belt too tightly fastened, which will cause discomfort, pain or even injury to the user. Post-stroke patients with impairments in their sensorimotor systems are especially prone to this kind of injury since they tend to be less sensitive to pain in their affected limb. Other potential problems include swelling due to the high pressure applied onto the skin and difficulties in muscle contraction. In light of the above problems, a wearable robotic device which attaches onto a user's limb by the tight fastening of a belt may not be suitable for use in long-term rehabilitation programs, such as those for post-stroke physical training. With regard to the slippage problem caused by sweating, it appears that none of the existing wearable robotic devices in the prior art has any mechanism addressing this problem.

Based on the above, the presently claimed invention is directed to overcoming the aforementioned problems by providing a wearable robotic device which has a bracing system with the functions of moisture and pressure management, hence able to minimize slippage between the device and the limb while avoiding high pressure from being exerted onto the user's limb by fastening belts.

### SUMMARY

It is an objective of the presently claimed invention to provide a wearable robotic device for rehabilitation purposes, which overcomes some of the limitations of the existing wearable robotic devices. More specifically, the presently claimed invention aims to minimize slippage between the device and the user's limb during physical training while avoiding the need to apply a high strapping pressure onto the user's limb. This ultimately leads to greater comfort for the user, especially for those who are engaged in long-term rehabilitation programs.

In accordance to the various embodiments of the presently claimed invention, the objective is achieved by including a bracing system in the wearable robotic device which has the functions of moisture and pressure management. Moisture management is achieved by having the bracing cushions made from a material which can draw moisture away from the user's skin; meanwhile, pressure management is achieved by having fastening belts which are elastic as well as having anti-slip borders on the bracing cushions and fastening belts.

### BRIEF DESCRIPTION OF THE DRAWINGS

Embodiments of the invention are described in more detail hereinafter with reference to the drawings, in which:

FIG. 1 shows a schematic representation of a wearable robotic device according to an embodiment of the present invention;

FIG. 2 shows the structure of a wearable robotic device with the fastening belts in an open position according to an embodiment of the present invention;

FIG. 3 shows a schematic representation of the structure of a textile bracing cushion; in particular FIG. 3A is a top view of the textile bracing cushion and FIG. 3B is a cross-sectional view of the textile bracing cushion;

FIG. 4 shows a schematic representation of the structure of a fastening belt;

FIG. 5 shows a wearable robotic device adapted for use across the wrist joint according to an embodiment of the present invention;

FIG. 6 shows a wearable robotic device adapted for use across the elbow joint according to an embodiment of the present invention;

FIG. 7 shows a wearable robotic device adapted for use across the knee joint according to an embodiment of the present invention; and

FIG. 8 shows a wearable robotic device adapted for use across the ankle joint according to an embodiment of the present invention.

### DETAILED DESCRIPTION

In the following description, the various designs of a wearable robotic device having a bracing system with moisture and pressure management functions are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

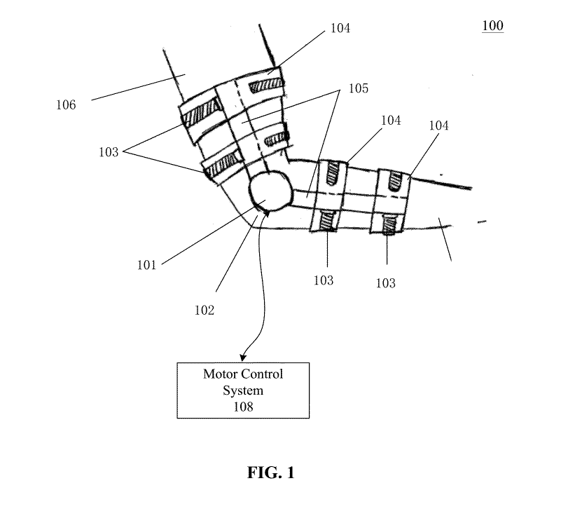

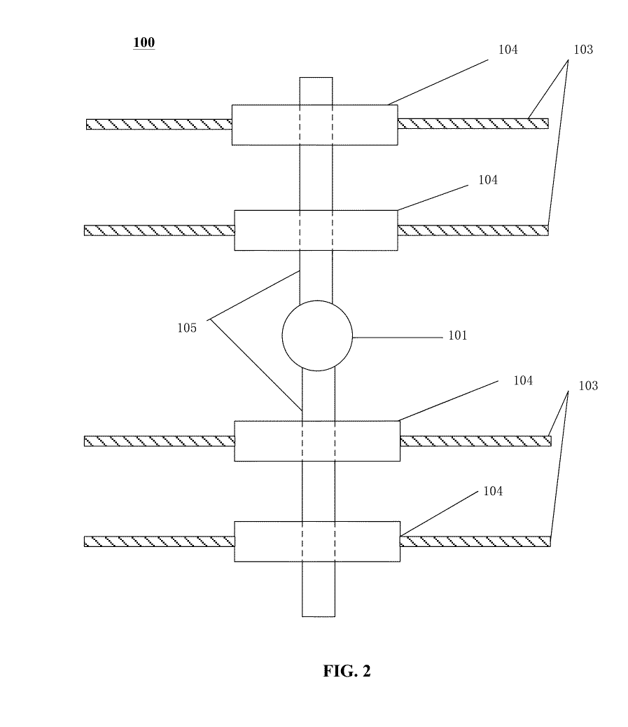

Referring to FIG. 1, there is shown a presently claimed wearable robotic device being worn across a joint 102. As shown in FIGS. 1 and 2, the wearable robotic device, in its basic form, includes a motor rotation system and a bracing system. The bracing system provides the mechanical framework for attaching the wearable robotic device onto the user's limb 106; meanwhile, the motor rotation system facilitates and controls the movement of the joint 102.

The motor rotation system comprises a motor 101 and a motor control system 108. The motor control system controls the rotation of the motor and limits its rotational range. An exemplary embodiment of the motor control system 108 is a micro-controller processor being in electronic communication with the motor 101, in which computer instructions are executed for controlling the rotation and limiting the rotational range of the motor 101. The motor rotation system is attached to the bracing system in such manner that the axis of rotation of the motor 101 coincides with the axis of rotation of the joint 102 of the limb 106.

The bracing system includes a framework of structural members 105 that is attachable to and detachable from the user's limb 106 by opening and closing of the fastening belts 103. The structural members 105 are arranged such that the rotation of the motor 101 can cause flexion or extension of the joint. The bracing system also includes one or more textile bracing cushions 104 attached to the framework of structural members 105. In the embodiment shown in FIGS. 1 and 2, four textile bracing cushions 104 are incorporated. The bracing system further includes one or more fastening belts 103 attached to the textile bracing cushions 104 such that the fastening belts 103 and the textile bracing cushions 104 in combination are capable of attaching the wearable robotic device 100 to the user's limb 106. In the embodiment shown in FIGS. 1 and 2, four sets of fastening belts 103 are respectively attached to the four textile bracing cushions 104.

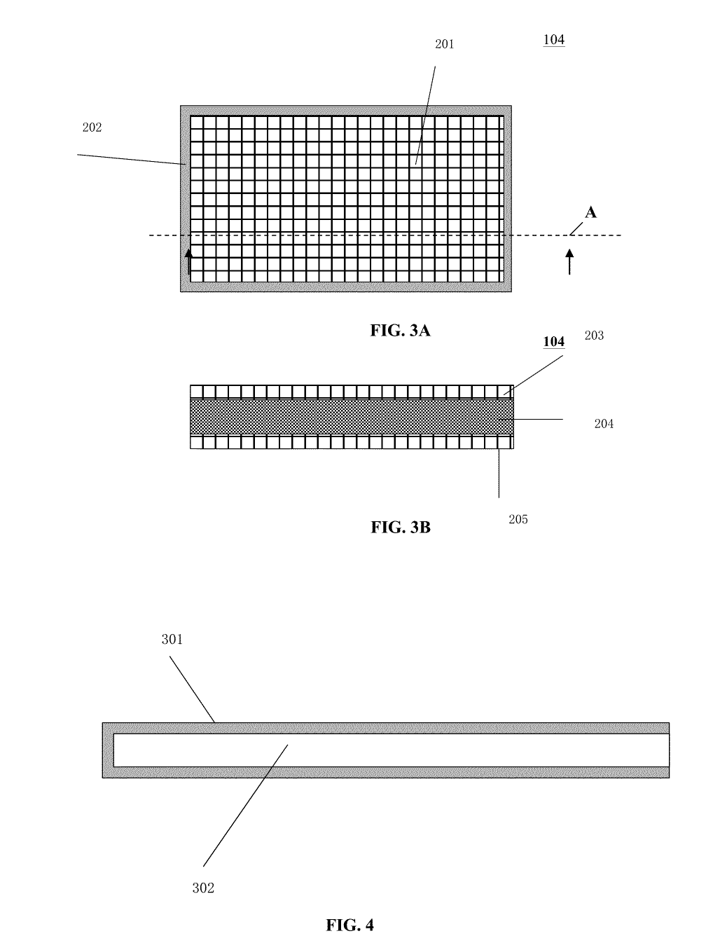

FIG. 3A shows a top view of a textile bracing cushion 104. As shown in FIG. 3A, each of the textile bracing cushions 104 has a main skin-contacting area 201 that is made of a material capable of drawing moisture away from the skin surface of a limb 106. Each textile bracing cushion 104 further comprises an anti-slip border 202 which is made of a resilient material having a higher coefficient of friction than the main skin-contacting area 201 and is disposed along the periphery of the textile bracing cushion 104 and is in contact with the skin surface of the limb 106. An example of the anti-slip border 202 on the textile bracing cushions 104 would be one that is made up of one or more elastic silicon strips.

FIG. 3B shows a cross-sectional view of a textile bracing cushion 104 across the plane A and viewed in the direction of the arrows. In accordance with the embodiment shown, the main skin-contacting area 201 is made of a material that has a sandwich structure comprising three layers. The first layer 203 has an inner surface, which is in contact with the skin surface of the limb 106 and an outer surface which is in contact with a second layer 204. In terms of material properties, the inner surface of the first layer 203 is hydrophobic while the outer layer is hydrophilic. The second layer 204 of the textile bracing cushion 104 is a piece of air mesh fabric for efficient ventilation of vapor and moisture. The second layer 204 is sandwiched between the first layer 203 and a third layer 205. The second layer 204 is in contact with an inner surface of the third layer 205; meanwhile, the outer surface of the third layer 205 is exposed in air. Further, the inner surface of the third layer 205 is hydrophobic, whereas the outer surface of the third layer 205 is hydrophilic.

The working principle is that any moisture, such as sweat, that is accumulated on the hydrophobic inner surface of the first layer 203 will be attracted to the hydrophilic outer surface of the first layer 203. Subsequently, the moisture that is now accumulated on the hydrophilic outer surface of the first layer 203 will be attracted, through the second layer 204 and the hydrophobic inner surface of the third layer 205, to the hydrophilic outer surface of the third layer 205, which is exposed in air. This mechanism of drawing moisture away from the skin surface keeps it relatively dry and therefore provides the “moisture management” function of the present invention.

FIG. 4 shows a fastening belt 103 according to an embodiment of the present invention. Each of the fastening belts 103 comprises an anti-slip border 301 and an elastic belt body 302. The elastic belt body 302 forms the base of the fastening belt 103, while the anti-slip border 301 is disposed along the periphery of the fastening belt to provide more friction between the fastening belt 103 and the skin surface of the limb 106. Both the anti-slip border 301 and the elastic belt body 302 are made of a resilient material, however, the anti-slip border 301 is made of a material which has a higher coefficient of friction than the elastic belt body 302. An example of the anti-slip border 301 on the textile bracing cushions 104 would be one that is made up of one or more elastic silicon strips. By having both the anti-slip border 301 and the elastic belt body 302 made of a resilient material, the length of the fastening belts 103 will be automatically adjusted to adapt to any changes in the shape and circumference of the limb 106 caused by muscle contraction or relaxation, and hence the pressure that is exerted onto the limb 106 by the fastening belts 103 will remain relatively constant.

Further, the anti-slip borders 202 and 301 on the textile bracing cushions 104 and the fastening belts 103, with their relatively high coefficients of friction, avoid the need to apply a high strapping pressure via the fastening belts 103 onto the user's limb 106 in order to fix the wearable robotic device 100 in place. This in turn lowers the risk of having the fastening belts 103 too tightly fastened and therefore reduces the chance of any resulting injuries.

In other words, both the resilient property of the fastening belts 103 and the anti-slip borders 202 and 301 can help prevent an excessively high pressure from being exerted onto the user's limb 106 and thereby achieving the “pressure management” function of the present invention.

As described above, the fastening belts 103 are attached to the textile bracing cushions 104 such that the fastening belts 103 and the textile bracing cushions 104 in combination are capable of attaching the wearable robotic device 100 onto the user's limb 106. The fastening belts 103 may be attached to the textile bracing cushions 104 by any means that is known in the art, such as sewing, riveting or threading the fastening belts through openings on the textile bracing cushions 104. Further, the fastening mechanism on the fastening belts 103 may be any fastening mechanism that is known in the art, such as the Velcro hook-and-loop fastening system or any type of buckles that are currently available.

According to another embodiment of the present invention, the wearable robotic device 100 further comprises a mechanical means disposed on the motor rotation system that limits the range of rotation of the motor 101. An example of such mechanical means is a set of mechanical stoppers, which physically prevent the flexion or extension of the joint 102 from exceeding a certain pre-set angle.

According to a further embodiment of the present invention, the wearable robotic device 100 also comprises a biosignal detecting unit configured to detect biosignals which are generated by the corresponding agonist muscle when the joint is being flexed or extended. Examples of such biosignals include electromyographic signals (EMG), electroencephalographic signals (EEG) and mechanomyographic signals (MMG). The motor control system 108 would then control the rotation of the motor 101 based on the biosignals detected by the biosignal detecting unit, for instance, in accordance with a set of pre-programmed instructions. The motor control system 108 may be configured as such that the angular velocity of the rotation of the motor 101 is proportional to the electromyographic activation level of the corresponding agonist muscle as detected by the biosignal detecting unit. In mathematical terms, it can be expressed in the form of Equation (1) below:

$$V ( t ) = \{ \begin{matrix}G \cdot {V}_{Max} \cdot {M}_{Flexion} , & During the flexion phase \\ G \cdot {V}_{Max} \cdot {M}_{Extesion} , & During the extension phase\end{matrix} \qquad ( 1 )$$

where V(t) is the angular velocity of the motor, G is the gain (e.g. ranging from 0 to 1) used to adjust the magnitude of the output velocity and V_{Max} is the pre-set maximum value of the angular velocity for the joint, for example, 20 degrees per second for the elbow joint during extension and flexion for a post-stroke patient. M_{Flexion} and M_{Extension} in Equation (1) are the normalized EMG envelopes of the corresponding agonist muscle during contraction.

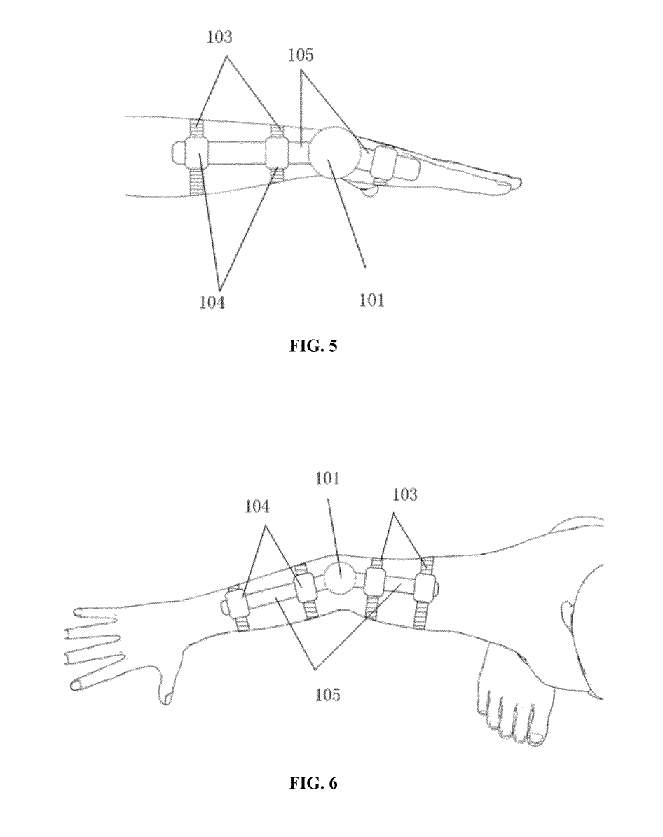

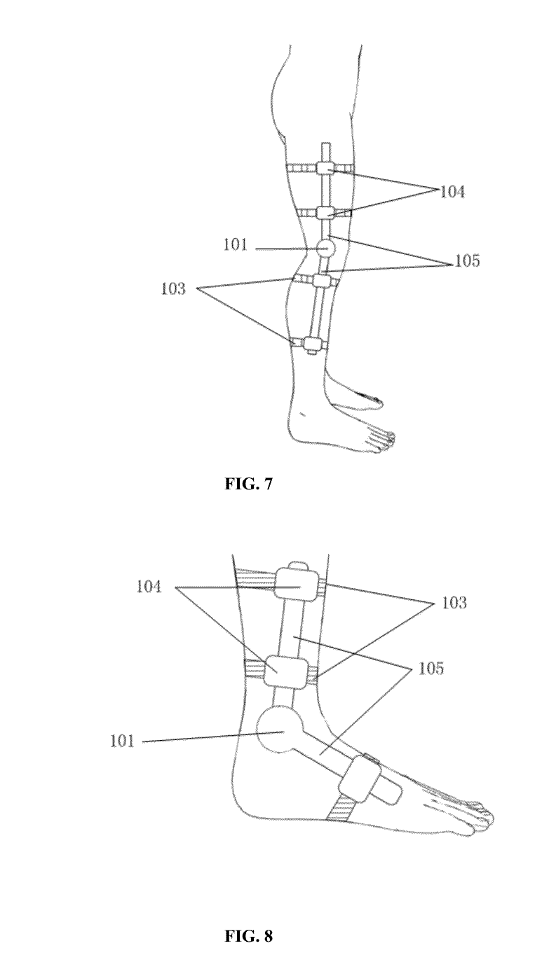

Referring to FIGS. 5 to 8, it is shown that the present invention may be adapted for use on various body joints by altering the shape, size and arrangement of the elements of the bracing system. As shown in FIGS. 5, 6, 7, and 8 respectively, the present invention may be adapted for use on the wrist joint, elbow joint, knee joint and ankle joint.

The embodiments of the motor control system disclosed herein may be implemented using general purpose or specialized computing devices, computer processors, or electronic circuitries including but not limited to micro-controller, digital signal processors (DSP), application specific integrated circuits (ASIC), field programmable gate arrays (FPGA), and other programmable logic devices configured or programmed according to the teachings of the present disclosure. Computer instructions or software codes running in the general purpose or specialized computing devices, computer processors, or programmable logic devices can readily be prepared by practitioners skilled in the software or electronic art based on the teachings of the present disclosure.

In some embodiments, the present invention includes computer storage media having computer instructions or software codes stored therein which can be used to program computers or microprocessors to perform any of the processes of the present invention. The storage media can include, but are not limited to, floppy disks, optical discs, Blu-ray Disc, DVD, CD-ROMs, and magneto-optical disks, ROMs, RAMs, flash memory devices, or any type of media or devices suitable for storing instructions, codes, and/or data.

The foregoing description of the present invention has been provided for the purposes of illustration and description. It is not intended to be exhaustive or to limit the invention to the precise forms disclosed. Many modifications and variations will be apparent to a practitioner skilled in the art.

The embodiments were chosen and described in order to best explain the principles of the invention and its practical application, thereby enabling others skilled in the art to understand the invention for various embodiments and with various modifications that are suited to the particular use contemplated. It is intended that the scope of the invention be defined by the following claims and their equivalence.

What is claimed is:

1. A wearable robotic device for rehabilitation training of a limb, comprising:
a motor rotation system; and
a bracing system;
wherein the motor rotation system comprises:
a motor;
a motor control system for controlling rotation of the motor and limiting rotational range of the motor;
wherein the motor rotation system is attached to the bracing system such that axis of rotation of the motor coincides with axis of rotation of a joint of the limb;
wherein the bracing system comprises:
a framework of one or more structural members that is attachable to and detachable from the limb, wherein arrangement of the structural members enables rotation of the motor to cause flexion and extension of the joint;
one or more textile bracing cushions attached to the framework of structural members and each having a main skin-contacting area, wherein the main skin-contacting area draws moisture away from skin surface of the limb that is in contact with the main skin-contacting area; and
one or more first anti-slip borders each disposed along at least a portion of periphery of each of the textile bracing cushions and in contact with the limb during use.

2. The wearable robotic device of claim 1, wherein the bracing system further comprises:
one or more fastening belts attached to each of the textile bracing cushions for attaching the wearable robotic device onto the limb and each having an elastic belt body; and
one or more second anti-slip borders each disposed along at least a portion of periphery of each of the fastening belts and in contact with the limb during use.

3. The wearable robotic device of claim 1, wherein the main skin-contacting area of each of the textile bracing cushions comprises:
a first layer comprising an inner surface being in contact with the limb during use, and an outer surface, wherein the inner surface of the first layer is hydrophobic and the outer surface of the first layer is hydrophilic;
a second layer comprising a piece of air mesh fabric and being in contact with the outer surface of the first layer; and
a third layer comprising an inner surface, which is in contact with the second layer, and an outer surface which is exposed in air, wherein the inner surface of the third layer is hydrophobic and the outer surface of the third layer is hydrophilic.

4. The wearable robotic device of claim 1, wherein each of the first anti-slip borders is made of a resilient material having a higher coefficient of friction than that of the main skin-contacting area.

5. The wearable robotic device of claim 2, wherein each of the second anti-slip borders is made of a resilient material having a higher coefficient of friction than that of the elastic belt body.

6. The wearable robotic device of claim 1, wherein each of the first anti-slip borders on each of the textile bracing cushions comprises one or more elastic silicon strips.

7. The wearable robotic device of claim 2, wherein each of the second anti-slip borders on each of the fastening belts comprises one or more elastic silicon strips.

8. The wearable robotic device of claim 1, further comprising one or more biosignal detecting units configured to detect biosignals which are generated by a corresponding agonist muscle when the joint is being flexed or extended;
wherein the motor control system controls the rotation of the motor based on the biosignals detected by the one or more biosignal detecting units.

9. The wearable robotic device of claim 5, wherein the one or more biosignal detecting units are configured to detect electromyographic signals.

10. The wearable robotic device of claim 5, wherein the one or more biosignal detecting units are configured to detect electroencephalographic signals.

11. The wearable robotic device of claim 5, wherein the one or more biosignal detecting units are configured to detect mechanomyographic signals.

12. The wearable robotic device of claim 6, wherein the motor control system is configured such that the angular velocity of the rotation of the motor is proportional to the electromyographic activation level of the corresponding agonist muscle as detected by the one or more biosignal detecting units.

13. The wearable robotic device of claim 1, further comprising a mechanical means disposed on the motor rotation system that limits rotational range of the motor.

14. The wearable robotic device of claim 10, wherein the mechanical means comprises of one or more mechanical stoppers which physically prevent flexion or extension of the joint of the limb from exceeding a pre-set angle.
